(19) United States
(12) Patent Application Publication
(10) Pub. No.: US 2009/0234359 A1
(43) Pub. Date: Sep. 17, 2009

(54) Mechanism for Osteosynthesis

(76) Inventors: Hidetoshi Onoue, Fukuoka (JP); Junji Ito, Osaka (JP); Toru Ikeda, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/085,868

(22) PCT Filed: Nov. 29, 2006
(86) PCT No.: PCT/JP2006/323785

§ 371 Date: May 30, 2008

(30) Foreign Application Priority Data
Dec. 1, 2005 (JP) 2005-348114

(51) Int. Cl. A61B 17/58; A61B 17/80

(52) U.S. Cl. 606/71; 606/282; 606/286

(57) ABSTRACT

A mechanism for osteosynthesis 1 is provided that is capable of finely adjusting the amount of movement of a fractured bone, quantifying the amount of the movement and can be used both in pulling the fractured bones apart from each other and pulling the fractured bones nearer toward each other. A first mechanism for osteosynthesis 1 of the present invention comprises a plate part 2 for reducing two or more fractured bones monolithically by fixing at least both ends of the plate part 2 on the fractured bones and a sliding part 25 for moving one fractured bone along a sliding elongate hole 3 formed in the plate part 2 so as to elongate from the one fractured bone toward another fractured bone, the sliding part 25 comprising a rack 31 formed on an inner surface of the sliding elongate hole so as to extend along a sliding direction and a pin inserted in the sliding elongate hole 3, the pin 4 having a head portion 41 having a pinion for engaging with the rack 31 and a base portion 45 to be inserted into the one fractured bone, wherein the sliding part 25 moves the one fractured bone slidably along the sliding elongate hole 3 by rotating the pin 4.

### BACKGROUND OF THE INVENTION

1. Field of the Invention

The present invention relates to a mechanism for osteosynthesis used for reducing and fixing fractured bones monolithically and promoting coaptation of the bone, in the event of fracture or osteotomy in diaphysis and epipysis.

2. Description of the Related Art

In case a part of a fractured bone is separated from the rest of the fractured bone, such an operation is performed as the separated part of the bone is replaced at the original position so as to promote coaptation of the fractured bones. One method of holding the separated part of a fractured bone at the original position for reduction is to keep the fractured bones together by means of a mechanism for osteosynthesis that employs such a member as bone plate that extends from the separated part of the fractured bone over the rest of the fractured bone. The separated part of the fractured bone, as it is kept together with the rest of the fractured bone monolithically, is coapted with the rest of the fractured bone and then the fracture is cured.

The mechanisms for osteosynthesis (a bone fixture) of the prior art include one that employs a bone plate constituted from a main plate section which has a through hole provided with a concave bearing surface and a head plate section that has a through hole provided with a concave bearing surface and female thread formed thereon (for example, Japanese Unexamined Patent Publication (Kokai) No. 2004-313514). The bone plate is fixed onto the bone by means of a bone screw inserted into the through hole. The bone screw has male thread formed in the head portion thereof, which is mated with the female thread formed in the through hole during use.

There is another mechanism for osteosynthesis that uses a bone plate having a first plate hole with thread formed over the entire circumference thereof and a second plate hole 38 without thread (for example, Japanese Unexamined Patent Publication (Kokai) No. 2003-509107). The bone plate is used with a lock screw that is inserted into the first plate hole 36 and a non-lock screw that is inserted into the second plate hole. The lock screw has thread formed on the head portion thereof that mates with the thread of the first plate hole.

When a mechanism for osteosynthesis that employs the bone plate is used, fractured bones must be secured in the same state as that before being fractured. However, fractured bones are often in a state different from that before fracture. For example, a tendon attached to the bone may pull a part of the fractured bone to cause it to overlap with the other part of the bone resulting in shortening dislocation, or the fractured parts of the bone may be pulled apart from each other resulting in a gap (separating dislocation). In case such a dislocation occurs, a bone plate is temporarily secured by means of forceps or the like while pulling the fractured bones apart from each other or nearer to each other with fingers to reduce the dislocation in the fractured bone.

However, the bone coaptation surgery based on this technique is not capable of reliably rectifying the dislocation in the fractured bone by means of fingers or making fine adjustment during rectification. In recent years, in addition, surgical techniques that require less incision into the skin is preferred in order to reduce invasion into the human body and reduce psychological burden on the patient, and it is very difficult to put the surgeon's fingers through a small incision opening and manipulate the fractured bones to precisely rectify the dislocation.

The bone coaptation surgery based on this technique also has such a problem that the amount of movement of the bone fragment required to precisely rectify the fractured bone cannot be quantitatively expressed, thus giving rise to the possibility that communication between the persons engaged in the surgical operation may become inaccurate.

In order to solve this problem, the mechanism for osteosynthesis described in Japanese Unexamined Patent Publication (Kokai) No. 2004-313514 is provided with a slide mechanism whereby the head of the bone screw moves sliding in an oblong hole to an extent corresponding to the degree of tightening the screw, thus enabling to finely adjust the amount of movement of the bone fragment. This slide mechanism is constituted from the oblong hole formed in a main plate section of the bone plate and the bone screw for sliding motion. The oblong hole has a concave bearing surface having a sloped surface that gradually becomes deeper with the distance from the head plate. As the bone screw is inserted into the oblong hole and is tightened so as to be secured in the bone, the force of tightening the bone screw causes the head of the bone screw to move down the sloped surface of the concave bearing surface, in other words slides away from the head plate. As a result, fractured bones can be pulled apart from each other. This mechanism of pulling apart would enable it to easily quantify the amount of movement of the fractured bone, and finely adjust the amount of movement of the bone fragment by controlling the tightening of the bone screw.

However, the mechanism of pulling apart disclosed in Japanese Unexamined Patent Publication (Kokai) No. 2004-313514 addresses shortening dislocation only, and is not applicable to separating dislocation. Also with this mechanism of pulling apart, a significant force of tightening is applied to a single bone sliding screw throughout the period from the start of moving the fractured bone by the slide mechanism to the completion of rectification and application of another bone screw for fixing. As a result, a strong force is required for tightening the bone sliding screw. Moreover, the force of tightening the screw produces strong stress concentrated in the bone in which the bone sliding screw is screwed in, and the concentrated stress may serve as a starting point of additional fracture.

### SUMMARY OF THE INVENTION

An object of the present invention is to provide a mechanism for osteosynthesis that enables it to finely adjust the amount of movement of a bone fragment, quantify the amount of movement and can be used both in pulling bone fragments apart from each other and nearer toward each other. Another object of the present invention is to provide a mechanism for osteosynthesis that is free from such problems as an excessive tightening force would be required for only a particular bone screw or an excessive load would be concentrated on the bone.

A first mechanism for osteosynthesis of the present invention comprises a plate part for reducing two or more fractured bones monolithically by fixing at least both ends of the plate part on the fractured bones and a sliding part for moving one fractured bone along a sliding elongate hole formed in the plate part so as to elongate from the one fractured bone toward another fractured bone, the sliding part comprising a rack formed on an inner surface of the sliding elongate hole so as to extend along a sliding direction and a pin inserted in the sliding elongate hole, the pin having a head portion having a pinion for engaging with the rack and a base portion to be inserted into the one fractured bone, wherein the sliding part moves the one fractured bone slidably along the sliding elongate hole by rotating the pin.

The mechanism for osteosynthesis is capable of both pulling bone fragments apart from each other and nearer toward each other by means of a gear mechanism. In particular, movements of bone ranging from a small distance of several millimeters to a relatively long distance of several centimeters can be accommodated by changing the length of the rack.

A second mechanism for osteosynthesis of the present invention comprises a plate part for reducing two or more fractured bones monolithically by fixing at least both ends of the plate part on the fractured bones and a sliding part for moving one fractured bone along a sliding elongate hole formed in the plate so as to elongate from the one fractured bone toward another fractured bone, the sliding part comprising a cam receiving portion formed on an inner surface of the sliding elongate hole and a pin inserted in the sliding elongate hole, the pin having a head portion having a cam for engaging with the cam receiving portion and a base portion to be inserted into the one fractured bone, wherein the sliding part moves the one fractured bone slidably along the sliding elongate hole by rotating the pin.

The mechanism for osteosynthesis makes it possible to easily pull bone fragments apart from each other and nearer toward each other by utilizing a cam mechanism. The bone plate enables it to use the cam mechanism of a relatively simple structure, and therefore helps reduce the manufacturing cost of the mechanism for osteosynthesis.

According to the first and second mechanism for osteosynthesis of present invention, it is made possible to finely adjust the amount of movement of the bone fragment by the amount of rotating the pin, and to easily quantify the amount of movement of the fractured bone in terms of the change in the position of the pin or the amount of rotating of the pin in the sliding elongate hole. The mechanism for osteosynthesis of the present invention is also capable of changing the direction of movement of the fractured bone by changing the direction of rotating the pin, and therefore can be used in both pulling the bone fragments nearer toward each other and pulling the bone fragments apart from each other.

Also the mechanism for osteosynthesis of present invention is capable of moving the fractured bone simply by rotating the pin, and therefore such problems can be avoided as an excessive tightening force would be required for only a particular bone screw or an excessive load would be concentrated on the bone.

### BRIEF DESCRIPTION OF THE DRAWINGS

FIG. 1 is a perspective view of a mechanism for osteosynthesis according to first embodiment of the present invention.

FIG. 2 is an enlarged front view of a sliding part of the mechanism for osteosynthesis according to the first embodiment of the present invention.

FIG. 3A is a perspective view of a pinion pin used in the sliding part of the mechanism for osteosynthesis according to the first embodiment of the present invention.

FIG. 3B is a perspective view of a bone pin that fixes the mechanism for osteosynthesis according to the present invention.

FIG. 3C is a perspective view of a bone screw that fixes the mechanism for osteosynthesis according to the present invention.

FIG. 3D is a perspective view of the bone screw that fixes the mechanism for osteosynthesis according to the present invention.

FIG. 4 is a front view of the mechanism for osteosynthesis according to the first embodiment of the present invention.

FIG. 5A is a sectional view showing a procedure to fix the mechanism for osteosynthesis according to the first embodiment of the present invention.

FIG. 5B is a sectional view showing a procedure to fix the mechanism for osteosynthesis according to the first embodiment of the present invention.

FIG. 5C is a sectional view showing a procedure to fix the mechanism for osteosynthesis according to the first embodiment of the present invention.

FIG. 5D is a sectional view showing a procedure to fix the mechanism for osteosynthesis according to the first embodiment of the present invention.

FIG. 5E is a sectional view showing a procedure to fix the mechanism for osteosynthesis according to the first embodiment of the present invention.

FIG. 5F is a sectional view showing a procedure to fix the mechanism for osteosynthesis according to the first embodiment of the present invention.

FIG. 6A is a front view of a mechanism for osteosynthesis according to second embodiment of the present invention.

FIG. 6B is a front view of a mechanism for osteosynthesis according to third embodiment of the present invention.

FIG. 6C is a front view of a mechanism for osteosynthesis according to fourth embodiment of the present invention.

FIG. 6D is a front view of a mechanism for osteosynthesis according to fifth embodiment of the present invention.

FIG. 6E is a front view of a mechanism for osteosynthesis according to sixth embodiment of the present invention.

FIG. 6F is a front view of a mechanism for osteosynthesis according to seventh embodiment of the present invention.

FIG. 6G is a front view of a mechanism for osteosynthesis according to eighth embodiment of the present invention.

FIG. 6H is a front view of a mechanism for osteosynthesis according to ninth embodiment of the present invention.

FIG. 6I is a front view of a mechanism for osteosynthesis according to tenth embodiment of the present invention.

FIG. 7 is a front view of a mechanism for osteosynthesis according to eleventh embodiment of the present invention.

FIG. 8 is an enlarged front view of a sliding part of the mechanism for osteosynthesis according to the eleventh embodiment of the present invention.

FIG. 9 is a perspective view of a cam pin used in the sliding part of the mechanism for osteosynthesis according to the eleventh embodiment of the present invention.

### BRIEF DESCRIPTION OF REFERENCE NUMERALS

1 Mechanism for osteosynthesis
10-12 Fractured bones
2 Bone plate
21 Diaphyseal fixing portion
23 Epiphysial fixing portion
25 Sliding part
3 Sliding elongate hole
31 Rack
32 Cam receiving portion
4 Pinion pin
41 Pinion (Pinion portion)
40 Cam pin
42 Cam (Cam portion)
45 Shaft
5 Hole with female thread
57 Female thread
50 Hole with spherical bearing surface
56 Spherical bearing surface
6 Auxiliary sliding elongate hole
8 Bone pin
81 Male thread on head of bone pin
9, 90 Bone screw
92 Flat head

### DETAILED DESCRIPTION OF THE INVENTION

### First Embodiment

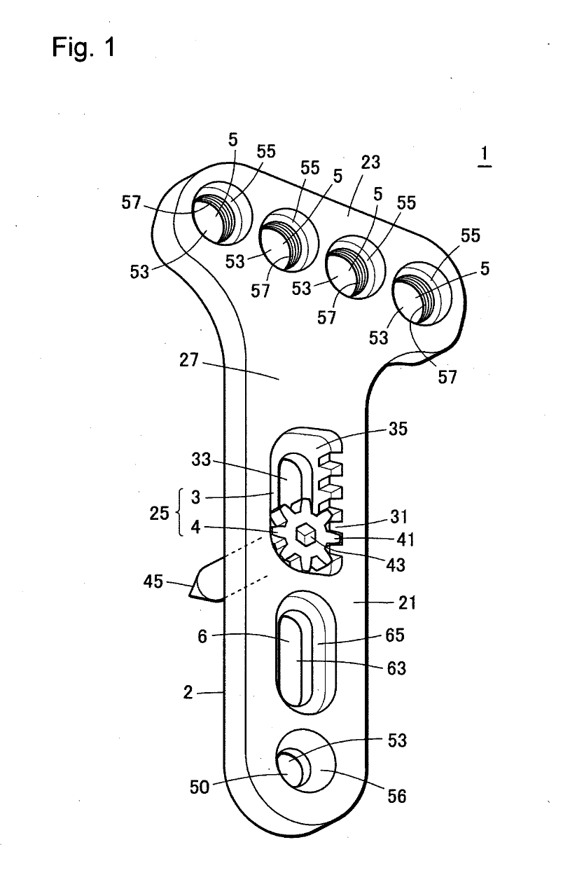

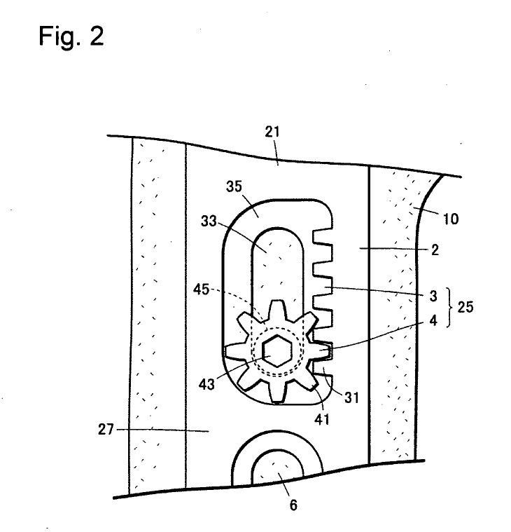

FIG. 1 and FIG. 2 show a mechanism for osteosynthesis (bone fixture) 1 used in fixation of a distal end of radius, among the mechanism for osteosynthesis provided with a gear mechanism according to the present invention. A plate part (a bone plate) 2 of the mechanism for osteosynthesis 1 of this embodiment is a substantially T-shaped plate extending longer in one direction, constituted from a slender diaphyseal fixing portion 21 and an epiphysial fixing portion 23 that is fixed on the top end of the diaphyseal fixing portion 21 in lateral direction.

The diaphyseal fixing portion 21 has a sliding elongate hole 3, an auxiliary sliding elongate hole 6 and a through hole 50 with a spherical bearing surface.

The sliding elongate hole 3 extends along the longitudinal direction of the bone plate 2 of the mechanism for osteosynthesis 1, and consists of a countersunk portion 35 of rectangular shape having four rounded corners formed on a surface 27 of the mechanism for osteosynthesis 1, a rack 31 formed along one of longer sides of the countersunk portion 3, and a through hole section 33 of oblong cross section formed on the back side of the mechanism for osteosynthesis 1.

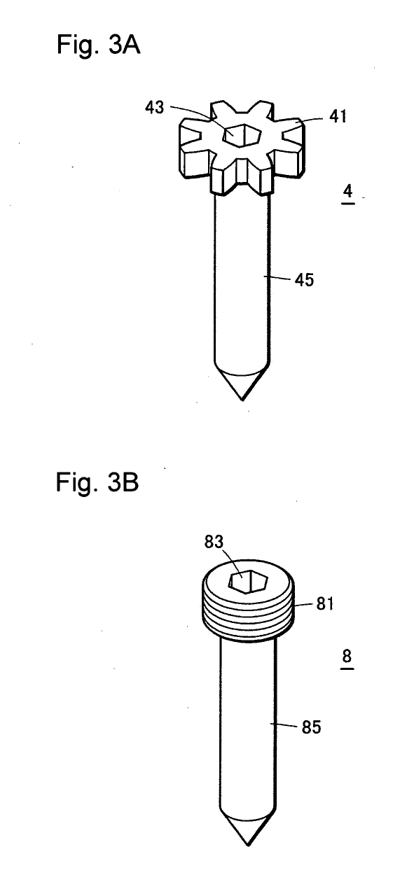

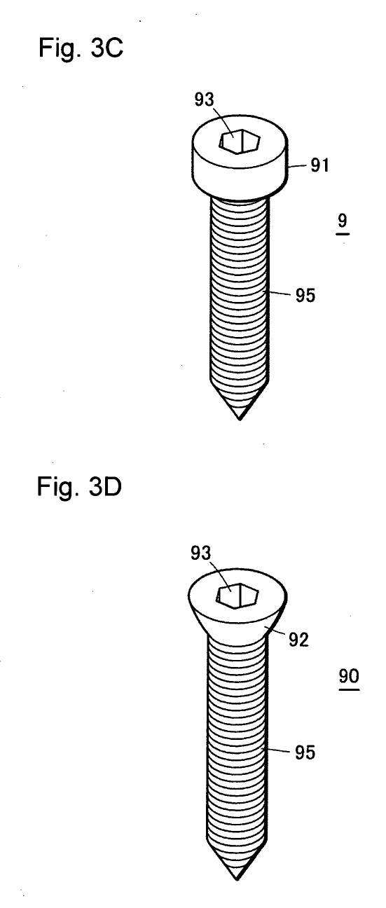

The mechanism for osteosynthesis 1 also includes a pin (a pinion pin 4) that has a gear-shaped head portion (a pinion portion 41) and a base portion (a shaft 45). The pinion pin 4 has the pinion portion 41 formed around the head portion that is inserted into the sliding elongate hole 3 so as to engage with the rack 31 as shown in FIG. 3A. Lower part of the pinion pin 4 is the cylindrical shaft 45 that passes the through hole section 33 of the sliding elongate hole 3 and is inserted into the bone. Tip of the shaft 45 is formed in a cone shape.

In a surgery to reduce a fractured bone by means of the mechanism for osteosynthesis 1 of the present invention, one part of the fractured bone can be moved along the longitudinal direction of the sliding elongate hole 3. The sliding part 25 that supports the bone is constituted by combining the sliding elongate hole 3 and the pinion pin 4.

The sliding part 25 that comprises the sliding elongate hole 3 and the pinion pin 4 will now be described in detail with reference to FIG. 2.

To install the sliding part 25 on a bone, first, the diaphyseal fixing portion 21 is placed at a predetermined position of one of the fractured bones, and the sliding elongate hole 3 is positioned on the predetermined position of the bone. Then the shaft 45 of the pinion pin 4 is inserted into the through hole 33 of the sliding elongate hole 3. While the shaft 45 is inserted into the bone to a predetermined position, it is necessary to insert the shaft 45 to such a depth as the pinion portion 41 of the pinion pin 4 makes contact with the countersunk surface 35. Thus the shaft 45 is rotatably fixed on the bone and the pinion portion 41 is engaged with the rack 31 of the sliding elongate hole 3.

In the sliding part 25 shown in FIG. 2, the shaft 45 is in the state of having been inserted into one of the fractured bones (a diaphyseal part 10), while the pinion portion 41 and the rack 31 being engaged with each other. As the pinion pin 4 is rotated, the pinion portion 41 rolls over the rack 31 and the fractured bone whereon the pinion pin 4 is fixed also moves sliding along the rack 31. In this way, the sliding part 25 can cause the bone to move sliding by rotating the pinion pin 4. The pinion pin 4 has a hexagonal socket 43 formed at the center of the head, and is driven to rotate by means of a hexagonal wrench that matches the hexagonal socket 43.

The countersunk portion 35 of the sliding elongate hole 3 is provided for the purpose of keeping the pinion portion 41 at a proper position in the direction of depth, so that the pinion portion 41 and the rack 31 engage with each other properly in the sliding elongate hole 3. Dimensions of the countersunk portion 35 in the longitudinal and lateral directions are set so as not to hamper the rotation and translating movement in the sliding direction of the pinion portion 41.

The through hole 33 of the sliding elongate hole 3 is where the shaft 45 of the pinion pin 4 passes through when the shaft 45 is inserted in the bone. Dimensions and shape of the through hole 33 are determined so as not to hamper the movement of the shaft 45 in the sliding direction when the sliding part 25 is manipulated.

The auxiliary sliding elongate hole 6 of the diaphyseal fixing portion 21 shown in FIG. 1 elongates parallel to the sliding elongate hole 3. The auxiliary sliding elongate hole 6 assists a sliding motion when the fractured bone is moved sliding by the sliding part 25.

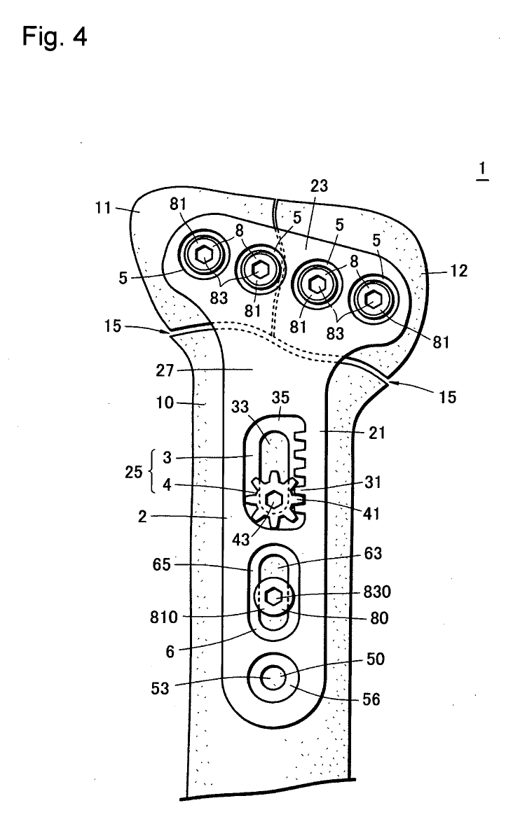

As shown in FIG. 4, the bone screw 9 is passed through the auxiliary sliding elongate hole 6, and is fixed by screwing on the fractured bone 10 whereon the pinion pin 4 is fixed. When the fractured bone 10 is moved by the sliding part 25, the bone screw 9 screwed into the fractured bone 10 moves sliding in the auxiliary sliding elongate hole 6. The motion of the bone screw 9 stabilizes the moving direction of the fractured bone 10. In other words, the auxiliary sliding elongate hole 6 has the function of guiding the moving direction of the fractured bone 10.

The auxiliary sliding elongate hole 6 can be used to temporarily fix the mechanism for osteosynthesis 1 and the fractured bone 10 to keep these members from departing from each other during the sliding movement. When manipulating the sliding part 25, the diaphyseal fixing portion 21 of the bone plate 2 is fixed onto the fractured bone 10 by means of the pinion pin 4. However, since the shaft 45 of the pinion pin 4 is not threaded, the shaft 45 is not capable of firmly fix the diaphyseal fixing portion 21 and the fractured bone 10 to each other. As a result, there is a possibility of the diaphyseal fixing portion 21 to come off the fractured bone 10. The diaphyseal fixing portion 21 can be suppressed from coming off the fractured bone 10 by holding the diaphyseal fixing portion 21 and the fractured bone 10 together by screwing using the auxiliary sliding elongate hole 6.

An ordinary bone screw 9 (for example, the bone screw 9 shown in FIG. 3C) is used for the auxiliary sliding elongate hole 6. The bone screw 9 may be one that is capable of self-tapping. The bone screw 9 is tightened to such an extent as the head of the bone screw 9 can slide in the auxiliary sliding elongate hole 6.

The auxiliary sliding elongate hole 6 has an oblong-shaped countersunk portion 65 that accommodates the head of the bone screw 9 and a through hole section 63 through which a shaft 95 of the bone screw 9 is inserted on the front surface 27 of the bone plate 2.

The mechanism for osteosynthesis 1 of the present invention keeps the fractured bones 10, 11, 12 together after reducing two or more fractured bones (FIG. 4 shows a state of the bone fractured into three fractured bones 10, 11, 12). Therefore the bone plate 2 of the mechanism for osteosynthesis 1 is fixed onto the fractured bones 10, 11, 12. The bone plate 2 shown in FIG. 1 has the female-threaded holes 5 that have female thread 57 (4 holes in the case shown) formed in the epiphysial fixing portion 23, and the trough hole 50 having a spherical bearing surface 56 (one hole in this drawing).

A shown in FIG. 1, the female-threaded hole 5 is formed in the epiphysial fixing portion 23 of the bone plate 2. The bone plate 2 is fixed onto the epipysis by inserting the special bone pin 8 shown in FIG. 3B in the female-threaded hole 5. The bone pin 8 has male thread 81 formed on the head that engages with a female thread 57, and the shaft 85. As the female thread 57 of the female-threaded hole 5 and the male thread 81 of the bone pin 8 engage with each other, the bone pin 8 is fixed in the bone plate 2. As a result, fractured bones 11, 12 wherein the shafts 85 of the bone pins 8 are inserted are stably held together without allowing swinging and swiveling.

Instead of the special bone pin 8 that can be used in the female-threaded hole 5, such a special bone pin that has male thread formed on the head and a shaft 85 having threaded base portion may also be used. Instead of the bone pin 8, such a special bone pin or a special bone screw may be used as the shaft of the bone pin or the special bone screw without male thread on the head is formed in two steps of the tip having small diameter and a portion right below the head having a large diameter, and male thread that engages with the female thread 57 of the female-threaded hole 5 is formed on the large diameter section of the shaft.

As shown in FIG. 1, the hole 50 with the spherical bearing surface is formed in the diaphyseal fixing portion 21 of the bone plate 2. The bone screw that is inserted in the hole 50 is capable of changing the angle of insertion as long as the head can swing in the spherical bearing surface 56. This makes it possible to insert the shaft of the bone screw into the bone at a desired angle when the shaft of the bone screw is fixed onto the bone. Therefore, such a bone screw that has a of the head portion shaped a appropriate configuration capable of swinging in the spherical bearing surface and has a the threaded shaft can be used in the hole 50 having the spherical bearing surface. An example of preferable bone screw is a bone screw 90 shown in FIG. 3D where the head is formed in the shape of a flat head 92 (a head having a flat top surface and a tapered bottom surface) and a shaft 95 having threaded base portion. A bone screw having a head with a spherical surface on the bottom is also preferably used in inserting in the hole 50 with the spherical bearing surface for fixing the bone plate 2.

In the bone plate 2 shown in FIG. 1, the epiphysial fixing portion 23 has the female-threaded holes 5 arranged in a row. However, the present invention is not limited to this configuration and the arrangement can be changed to two rows or random arrangement, which may be selected in accordance to the dimensions and shape of the epipysis of the patient and the condition of fracture at the epipysis.

The bone plate 2 of the mechanism for osteosynthesis 1 is integrally formed from a metal of high biocompatibility such as titanium alloy, cobalt-chromium alloy or stainless steel.

The pinion pin 4, the bone pin 8 and the bone screws 9, 90 are formed from metal of high biocompatibility such as titanium alloy or cobalt-chromium alloy.

As shown in FIG. 1 to FIG. 4, the pinion pin 4, the bone pin 8 and the bone screws 9, 90 have hexagonal sockets 43, 83, 93 formed in the top face of the head thereof, and can be rotated by means of hexagonal socket wrench. The hexagonal sockets 43, 83, 93 may be replaced with sockets of other shape such as slit, cross slot, square socket or hexa-lobed socket. Hexagonal socket and hexa-lobed socket are capable of reliably transferring a high torque and are preferably used.

A procedure of fixing the fractured bone by using the mechanism for osteosynthesis 1 of the present invention will now be described.

FIG. 4 shows the state of the mechanism for osteosynthesis 1 being fixed onto fractured portion of the distal end of radius, before the fractured bone is reduced. In this case of fracture, the radius is fractured into a diaphyseal part 10 and two epiphysial parts 11, 12 located at the distal side, while the diaphyseal part 10 and the epiphysial parts 11, 12 of the fractured bone have undergone separating dislocation. As a result, there is a gap 15 generated between the diaphyseal part 10 and the epiphysial parts 11, 12 of the fractured bone, and therefore the epiphysial parts 11, 12 are pulled up toward the diaphyseal part 10.

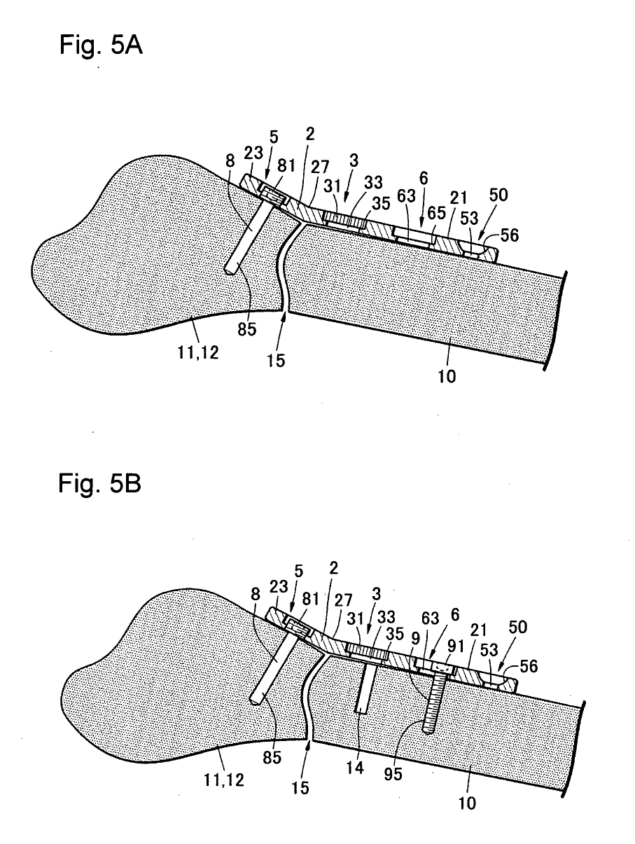

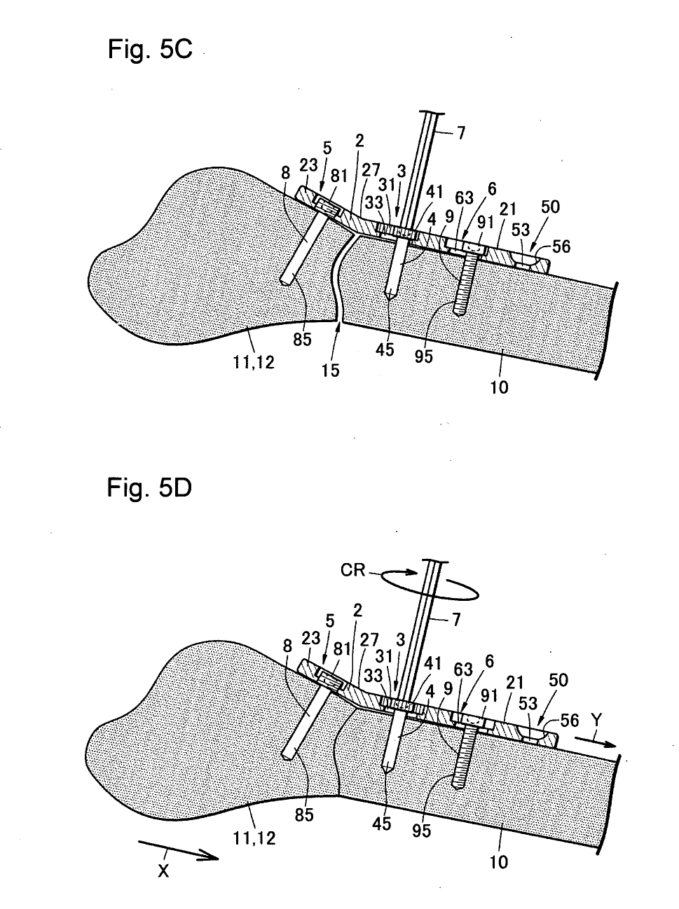

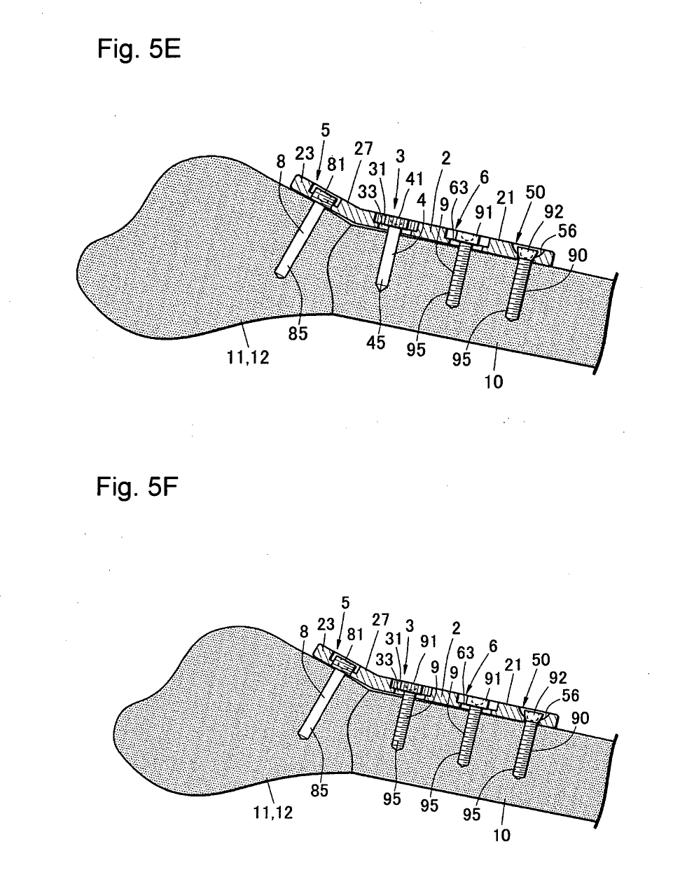

The state of fixing the mechanism for osteosynthesis 1 as shown in FIG. 4 is achieved by the procedure shown in FIG. 5A to FIG. 5C, and the subsequent operations of reducing and fixing the fractured bones are carried out in the procedure shown in FIG. 5D to FIG. 5F.

FIG. 5A shows a step of inserting the bone pins 8 having male thread 81 formed on the head thereof through the four female-threaded holes 5 formed in the epiphysial fixing portion 21 of the bone plate 2 and fixing the bone plate 2 onto the epiphysial parts 11, 12. The epiphysial fixing portion 23 of the bone plate 2 is warped with respect to the diaphyseal fixing portion 21, so as to match the shape of the distal side of the radius that swells from the diaphyseal part 10 toward the epiphysial part 11, 12 as shown in the drawing.

FIG. 5B shows a step of screwing the bone screw 9 through the auxiliary sliding elongate hole 6 of the diaphyseal fixing portion 21 so as to fix onto the diaphyseal part 10 of the fractured bone. The bone screw 9 is tightened to such an extent that the bone plate 2 of the mechanism for osteosynthesis 1 would not be lifted from the diaphyseal part 10 and, at the same time, the bone screw 9 can move sliding in the auxiliary sliding elongate hole 6. Since this is a case of fracture with separating dislocation, the bone screw 9 is set at a position in the auxiliary sliding elongate hole 6 away from the epiphysial parts 11, 12, namely at a position nearer to the proximal side.

A prepared hole 14 for inserting the pinion pin 4 is formed in the diaphyseal part 10 of the fractured bone at a position corresponding to the through hole 33 of the sliding elongate hole 3 of the bone plate 2. The prepared hole 14 is, similarly to the bone screw 9, set at a position in the sliding elongate hole 3 away from the epiphysial parts 11, 12, namely at a position nearer to the proximal side.

Then the pinion pin 4 is set in the sliding elongate hole 3 as shown in FIG. 5C, thereby assembling the sliding part 25. The assembling operation is carried out by inserting the shaft 45 of the pinion pin 4 from the through hole section 33 of the sliding elongate hole 3 into the prepared hole 14 of the diaphyseal part 10 of the fractured bone, and engaging the pinion portion 41 formed on the head of the pinion pin 4 with the rack 31 of the sliding elongate hole 3.

When the sliding part 25 has been assembled, a hexagonal socket wrench 7 is put into the hexagonal socket 43 of the pinion pin 4 as shown in FIG. 5D, and the pinion pin 4 is rotated clockwise in a CR direction. The pinion portion 41 of the pinion pin 4 engages with the rack 31 and moves toward the epiphysial parts 11, 12 of the fractured bone in the sliding elongate hole 3. This means that the pinion pin 4 and the bone plate 2 move with respect to each other. In actuality, since the pinion pin 4 is fixed in the diaphyseal part 10 of the fractured bone, the bone plate 2 moves toward the proximal side of the diaphyseal part 10 in the direction indicated by arrow Y. Also the epiphysial parts 11, 12 fixed by the bone pin 8 onto the epiphysial fixing portion 23 of the bone plate 2 move together with the bone plate 2 toward the diaphyseal part 10 in the direction indicated by arrow X. The pinion portion 41 is rotated until the epiphysial parts 11, 12 make contact with the diaphyseal part 10 to eliminate the gap 15, or until the gap between the diaphyseal part 10 and the epiphysial parts 11, 12 of the bone is reduced to a predetermined size. FIG. 5D shows a case of reduction conducted to eliminate the gap 15.

When the fractured bone has been reduced as shown in FIG. 5D, the diaphyseal fixing portion 21 of the bone plate 2 is fully fixed onto the diaphyseal part 10 of the fractured bone as shown in FIG. 5E. First, the bone screw 9 that has been lightly tightened in the auxiliary sliding elongate hole 6 to such an extent that allows sliding thereof is tightened firmly to prevent it from sliding, so as to temporarily fix the bone plate 2 onto the diaphyseal part 10 thereby making the subsequent fixing operation easier. Then a prepared hole is formed in the diaphyseal part 10 at a position corresponding to the hole 50 with the spherical bearing surface of the bone plate 2. The prepared hole may be formed at a desired angle with respect to the diaphyseal part 10 (approximately 90 degrees from an axis of the diaphyseal part in the case shown). The flat head bone screw 90 is screwed into the prepared hole so as to fully fix the diaphyseal fixing portion 21 and the diaphyseal part 10 together. When screwing the bone screw 90 into the prepared hole, it is preferable to employ the so-called self-tapping operation in which the shaft 95 forms mating thread while screwing into the diaphyseal part 10, which achieves firm connection between the bone screw 90 and the diaphyseal part 10.

The pinion pin 4 may thereafter be left to remain in the patient's body. However, it is preferable to replace the pinion pin 4 with the bone screw 9 as shown in FIG. 5F, which achieves stronger fixing of the fractured bones by means of the bone plate 2. As the hole remains in the b diaphyseal part 10 of the fractured bone where the shaft 45 of the pinion pin 4 has been inserted, it is preferable to use this hole for inserting the bone screw 9 to fix the sliding elongate hole 3 and the diaphyseal part 10.

Through the series of steps described above, the mechanism for osteosynthesis 1 of the present invention can keep the epiphysial parts 11, 12 that have been separated by fracture and the diaphyseal part 10 together.

Replacement of the pinion pin 4 with the bone screw 9 may also be done before screwing the flat head bone screw 90 into the diaphyseal part 10 of the fractured bone in the step shown in FIG. 5E. For example, the pinion pin 4 may be replaced with the bone screw 9 after tightening the bone screw 9 in the auxiliary sliding elongate hole 6 thereby temporarily fixing the bone plate 2 with the diaphyseal part 10 in the step shown in FIG. 5E. This procedure is preferable as the force of fixing can be increased because the bone plate 2 remains temporarily fixed by the two bone screws 9 while drilling the prepared hole in the diaphyseal part 10 for inserting the bone screw 90 at a position corresponding to the hole 50 with the spherical bearing surface of the bone plate 2.

While FIGS. 5A to 5F show the steps of pulling the fractured bones (which are separated) toward each other in the case of fracture with separating dislocation, the present invention can be applied also to a case of fracture with shortening dislocation.

The fractured bones can be pulled apart from each other in the case of fracture with shortening dislocation, simply by changing three steps among those shown in FIGS. 5A to 5F:

(I) Change the position where the bone screw 9 is set in the auxiliary sliding elongate hole 6 in the step shown in FIG. 5B to a position in the auxiliary sliding elongate hole 6 nearer to the epiphysial parts 11, 12, namely at a position nearer to the distal side;
(II) Form the prepared hole for inserting the pinion pin 4 shown in FIG. 5B at a position in the sliding elongate hole 3 nearer to the epiphysial parts 11, 12, namely at a position nearer to the distal side;
(III) Change the direction of rotating the pinion pin 4 in the step shown in FIG. 5D to counterclockwise, so as to move the pinion portion of the pinion pin 4 in the sliding elongate hole 3 in a direction of moving away from the epiphysial parts 11, 12. With these changes, the diaphyseal part 10 and the epiphysial parts 11, 12 can be pulled apart from each other by using the same mechanism for osteosynthesis 1.

The present invention makes it possible, by providing the pinion pin 4 and the sliding elongate hole 3, to reduce the fractured bone by rotating the pinion pin 4. This makes it easier to reduce the fractured bone and makes it possible to easily quantify the amount of movement of the fractured bone. Also because the pinion pin 4 can be rotated by means of a wrench, the operation of rotating the pinion pin 4 is made easier and the reduction of the fractured bone can be done reliably even when the incision opening is small. The present invention can be applied to pulling bone fragments both apart from each other and nearer toward each other simply by changing the direction of rotating the pinion pin 4.

### Second Embodiment

The mechanism for osteosynthesis of the present invention can be made in such a form that can be applied to the fracture of various bones such as humerus, forearm (including radius and ulna), vertebra, femur, crus (including tibia and fibula), phalanges of hand and phalanges of foot, in addition to radius as in the first embodiment. One form of the mechanism for osteosynthesis of the present invention will be described below.

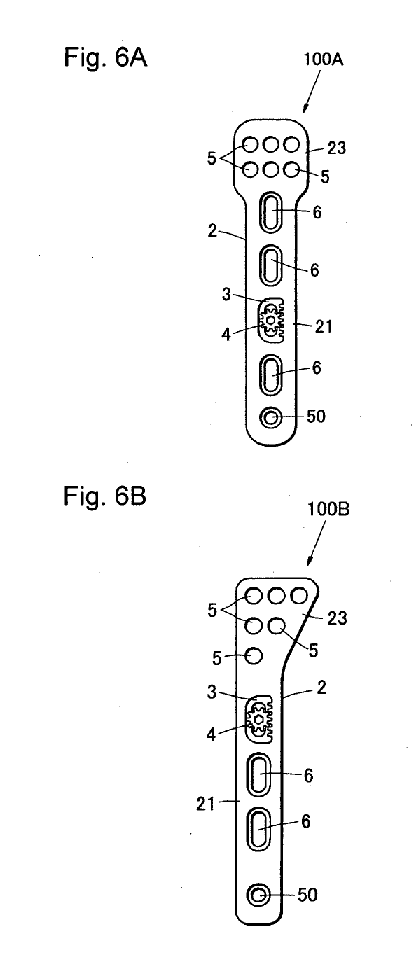

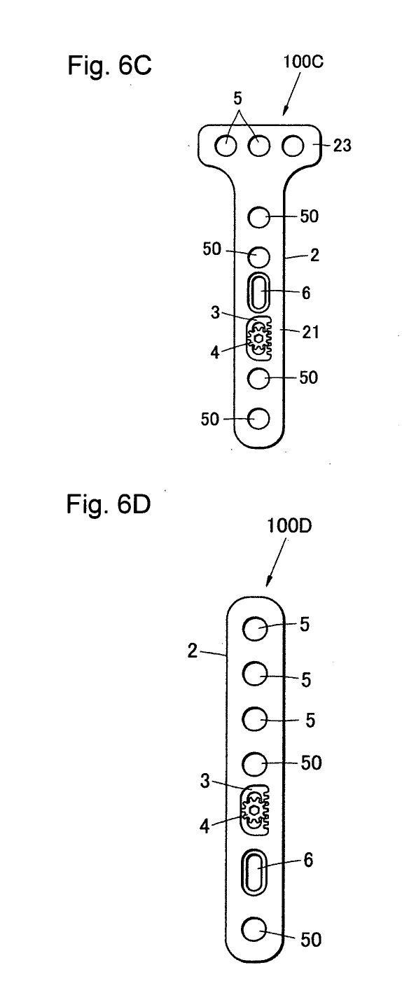

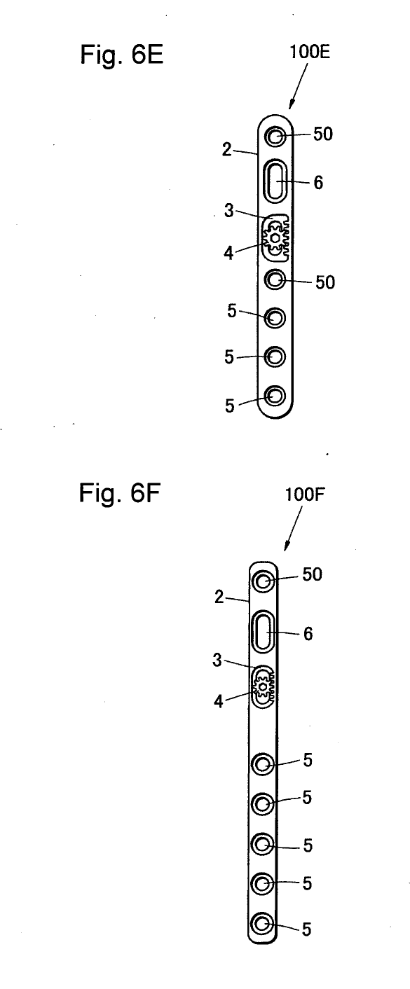

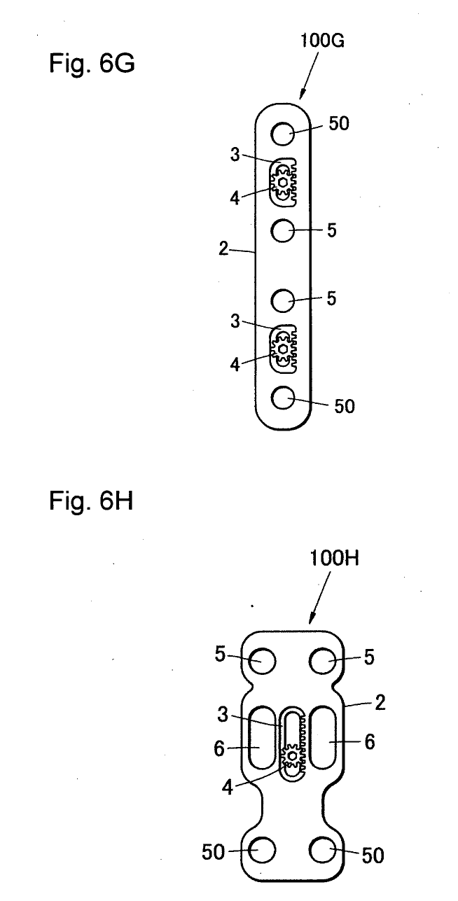

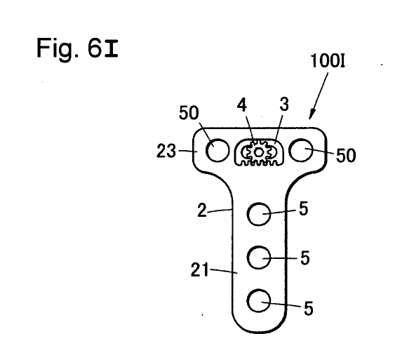

FIG. 6A shows a mechanism for osteosynthesis 100A to be used in proximal side of forearm bone where the epiphysial fixing portion 23 of the bone plate 2 is fixed onto the epipysis of the humerus and the diaphyseal fixing portion 21 of the bone plate 2 is fixed onto the diaphysis of the humerus. The mechanism for osteosynthesis 100A comprises the bone plate 2, the sliding elongate hole 3 formed in the bone plate 2, a plurality of female-threaded holes 5 (arranged in 2 rows and 3 columns in rectangular configuration in this example), a plurality of auxiliary sliding elongate holes 6 (three in this example), the hole 50 with the spherical bearing surface and the pinion pin 4 to be fitted in the sliding elongate hole 3.

Method of using the mechanism for osteosynthesis 100A shown in FIG. 6A is similar to that of the mechanism for osteosynthesis 1 of the first embodiment used for radius. The method comprises (1) fixing the epiphysial fixing portion 23 of the bone plate 2 on the epiphysial part of the fractured bone; (2) fixing the auxiliary sliding elongate hole 6 with the bone screw and the sliding elongate hole 3 with the pinion pin 4 on the diaphyseal part of the fractured bone; (3) rotating the pinion pin 4 so as to pull the diaphyseal part and the epiphysial parts toward each other or away from each other thereby to reduce the fractured bone; and (4) inserting a bone screw 90 with a tapered head in the hole 50 with the spherical bearing surface and screwing bone screw 90 in the diaphyseal part to fix the bone plate on the diaphyseal part.

The mechanism for osteosynthesis of this embodiment is suited for fixing fractured bone in the proximal side of humeral bone. By adjusting the length of the fractured bone by rotating the pinion pin 4, it is made easy to reduce the fractured bone. In addition, it is made easier to quantify the amount of movement of the fractured bone. Also because the pinion pin 4 is rotated with a wrench, the pinion pin 4 can be easily operated and rotated even when the incision opening is small. Also the present invention is applicable to both pulling the bone fragments apart from each other and pulling the bone fragments nearer toward each other simply by changing the direction of rotating the pinion pin 4.

### Third Embodiment

FIG. 6B shows a mechanism for osteosynthesis 100B to be used in distal side of femur, where the epiphysial fixing portion 23 of the bone plate 2 is fixed onto the epiphysial part of the femur and the diaphyseal fixing portion 21 of the bone plate 2 is fixed onto the diaphysis of the femur. The mechanism for osteosynthesis 100B comprises the bone plate 2, the sliding elongate hole 3 formed in the bone plate 2, a plurality of female-threaded holes 5 (6 holes are arranged in triangular configuration in this example), a plurality of auxiliary sliding elongate holes 6 (two in this example), the hole 50 with the spherical bearing surface and the pinion pin 4 to be fitted in the sliding elongate hole 3.

Method of using the mechanism for osteosynthesis 100B shown in FIG. 6B is similar to that of the first and second embodiments. The method comprises (1) fixing the epiphysial fixing portion 23 on the epiphysial part of the fractured bone; (2) fixing the auxiliary sliding elongate hole 6 with the bone screw and the sliding elongate hole 3 with the pinion pin 4 on the diaphyseal part of the fractured bone; (3) rotating the pinion pin 4 so as to pull the diaphyseal part and the epiphysial part toward each other or away from each other and reduce the fractured bone; and (4) inserting the bone screw 90 with the tapered head in the hole 50 with the spherical bearing surface and screwing bone screw 90 in the diaphyseal part to fix the bone plate on the diaphyseal part.

The mechanism for osteosynthesis of this embodiment is suited for fixing fractured bone in the distal side of femur. By adjusting the length of the fractured bone by rotating the pinion pin 4, it is made easy to reduce the fractured bone. In addition, it is made easier to quantify the amount of movement of the fractured bone. Also because the pinion pin 4 is rotated with a wrench, the pinion pin 4 can be easily rotated even when the incision opening is small. Also the present invention is applicable to both pulling the bone fragments apart from each other and pulling the bone fragments nearer toward each other simply by changing the direction of rotating the pinion pin 4.

### Fourth Embodiment

FIG. 6C shows a mechanism for osteosynthesis 100C to be used in tibia where the epiphysial fixing portion 23 of the bone plate 2 is fixed onto the epipysis of the tibia and the diaphyseal fixing portion 21 of the bone plate 2 is fixed onto the diaphysis of the tibia. The mechanism for osteosynthesis 100C comprises the bone plate 2, the sliding elongate hole 3 formed in the bone plate 2, a plurality of female-threaded holes 5 (3 holes are arranged in a row in this example), the auxiliary sliding elongate hole 6, the holes 50 with the spherical bearing surface (two on either side of the sliding elongate hole 3, 4 in all in this example) and the pinion pin 4 to be fitted in the sliding elongate hole 3.

Method of using the mechanism for osteosynthesis 100C shown in FIG. 6C is similar to that of the first to third embodiments. The method comprises (1) fixing the epiphysial fixing portion 23 of the bone plate 2 on the epiphysial part of the fractured bone; (2) fixing the auxiliary sliding elongate hole 6 with the bone screw and the sliding elongate hole 3 with the pinion pin 4 on the diaphyseal part of the fractured bone; (3) rotating the pinion pin 4 so as to pull the diaphyseal part and epiphysial part toward each other or away from each other thereby to reduce the fractured bone; and (4) inserting the bone screw 90 with tapered head in the hole 50 with the spherical bearing surface and screwing bone screw 90 in the diaphyseal part to fix the bone plate on the diaphyseal part.

The mechanism for osteosynthesis of this embodiment is suited for fixing fractured bone in the tibia. By adjusting the amount of movement of the bone fragment by rotating the pinion pin 4, it is made easy to reduce the fractured bone. In addition, it is made easier to quantify the amount of movement of the fractured bone. Also because the pinion pin 4 is rotated with a wrench, the pinion pin 4 can be easily manipulated and rotated even when the incision opening is small. Also the present invention is applicable to both pulling the bone fragments apart from each other and pulling the bone fragments nearer toward each other simply by changing the direction of rotating the pinion pin 4.

### Fifth to Seventh Embodiments

FIG. 6D shows a mechanism for osteosynthesis 100D to be used in a disphysis of humerus, with each end portion of the bone plate 2 being fixed onto each of two fractured disphysial parts. The mechanism for osteosynthesis 100D comprises the sliding elongate hole 3, the pinion pin 4, a plurality of female-threaded holes 5 (3 holes in this example), the auxiliary sliding elongate hole 6 and the holes 50 with the spherical bearing surface (two in this example).

FIG. 6E shows a mechanism for osteosynthesis 100E to be used in a disphysis of ulna, with each end portion of the bone plate 2 being fixed onto each of two fractured disphysial parts. The mechanism for osteosynthesis 100E comprises the bone plate 2, the sliding elongate hole 3 formed in the bone plate 2, a plurality of female-threaded holes 5 (3 holes in this example), the auxiliary sliding elongate hole 6, the holes 50 with the spherical bearing surface (two in this example) and the pinion pin 4 to be fitted in the sliding elongate hole 3.

FIG. 6F shows a mechanism for osteosynthesis 100F to be used in a disphysis of humerus, with each end portion of the bone plate 2 being fixed onto each of two fractured disphysial parts. The mechanism for osteosynthesis 100F comprises the sliding elongate hole 3, the pinion pin 4, a plurality of female-threaded holes 5 (5 holes in this example), the auxiliary sliding elongate hole 6 and the hole 50 with the spherical bearing surface (one in this example).

Methods of using the mechanisms for osteosynthesis 100D, 100E and 100F shown in FIGS. 6D to 6F are similar to those of the first to fourth embodiments. The methods comprise (1) fixing one end of the bone plate 2 on one fractured disphysial part by using the female-threaded hole 5 formed in the one end of the bone plate 2; (2) inserting a bone screw in the auxiliary sliding elongate hole 6 and screwing bone screw in the other fractured disphysial part, and inserting the pinion pin 4 in the sliding elongate hole 3 and in the other fractured disphysial part, in order to fix the bone plate to the other fractured disphysial part; (3) rotating the pinion pin 4 so as to pull the two fractured disphysial parts toward each other or away from each other thereby to reduce the fractured bone; and (4) inserting the bone screw 90 with the tapered head in the hole 50 with the spherical bearing surface and screwing bone screw 90 in the other fractured disphysial part to fix the bone plate on the other fractured disphysial part.

The mechanism for osteosynthesis of this embodiment is suited for fixing fractured bone in the disphysis of humerus, the disphysis of ulna and the disphysis of humerus. Since adjustment of the length of the fractured bone can be achieved by rotating the pinion pin 4, reduction of the fractured bone is easy. In addition, it is made easier to quantify the amount of movement of the fractured bone. Also because the pinion pin 4 is rotated with a wrench, the pinion pin 4 can be easily manipulated and rotated even when the incision opening is small. Also the present invention is applicable to both pulling the bone fragments apart from each other and pulling the bone fragments nearer toward each other simply by changing the direction of rotating the pinion pin 4.

### Eighth Embodiment

FIG. 6G shows a mechanism for osteosynthesis 100G to be used in a disphysis, and is suited for pulling three fractured disphysial parts to each other. The mechanism for osteosynthesis 100G comprises the bone plate 2, two sliding elongate holes 3 formed in the bone plate 2, two female-threaded holes formed between the sliding elongate holes 3, the holes 50 with the spherical bearing surface formed on either end of the bone plate 2 and two pinion pins 4 to be fitted in the sliding elongate holes 3. With the mechanism for osteosynthesis 100G, each end of the bone plate 2 is fixed respectively onto two fractured disphysial parts located on either side among three fractured disphysial parts, and a center portion of the bone plate 2 is fixed onto a fractured disphysial parts located at the center by using the female-threaded hole 5.

Method of using the mechanism for osteosynthesis 100G shown in FIG. 6G is similar to those of the first to seventh embodiments. The method comprises (1) fixing the bone plate 2 onto the fractured disphysial parts located at the center of the three fractured disphysial parts by using the female-threaded hole 5 formed near the center of the bone plate 2; (2) inserting two pinion pins 4 in the two sliding elongate holes 3 respectively and in the respective fractured disphysial parts located on both side of the three fractured disphysial parts; (3) rotating the two pinion pins 4 so as to pull the three fractured disphysial parts toward each other or away from each other and reduce the fractured bone; and (4) inserting the bone screws 90 with the tapered head in the holes 50 with the spherical bearing surface and screwing bone screws 90 in the respective fractured disphysial parts located on both side to fix the each end of the bone plate on the respective fractured disphysial parts.

Thus the bone pins suitable for the reduction of various conditions of bone fracture can be provided by changing the numbers of pinion pins 4 and the sliding elongate holes 3.

The mechanism for osteosynthesis of this embodiment can be preferably used in fixing fractured bone in the diaphysis of various bones by changing the dimensions thereof. By adjusting the length of the fractured bone by rotating the pinion pin 4, it is made easy to reduce the fractured bone. In addition, it is made easier to quantify the amount of movement of the fractured bone. Also because the pinion pin 4 is rotated with a wrench, the pinion pin 4 can be easily rotated even when the incision opening is small. Also the present invention is applicable to both pulling the bone fragments apart from each other and pulling the bone fragments nearer toward each other simply by changing the direction of rotating the pinion pin 4.

### Ninth Embodiment

FIG. 6H shows a mechanism for osteosynthesis 100H to be used in vertebra, with each end portion of the bone plate 2 being fixed onto each part of fractured vertebra. The mechanism for osteosynthesis 100H comprises the bone plate 2, the sliding elongate hole 3 formed in the bone plate 2, a plurality of female-threaded holes 5 (two in this example), the holes 50 with the spherical bearing surface (two in this example), a plurality of auxiliary sliding elongate holes 6 (two arranged in parallel to the sliding elongate hole 3 in this example) and the pinion pin 4 to be fitted in the sliding elongate hole 3.

Method of using the mechanism for osteosynthesis 100H shown in FIG. 6H is similar to those of the first to third embodiments. The method comprises (1) fixing one end of the bone plate 2 onto one fractured vertebral part by using the female-threaded hole 5 formed in the one end of the bone plate 2; (2) inserting a bone screw in the auxiliary sliding elongate hole 6 and screwing bone screw in the other fractured vertebral part, and

inserting the pinion pin 4 in the sliding elongate hole 3 and in the other fractured vertebral part, in order to fix the bone plate to the other fractured vertebral part; (3) rotating the pinion pin 4 so as to pull the two fractured vertebral parts toward each other or away from each other to reduce the fractured bone; and (4) inserting the bone screw 90 with the tapered head in the hole 50 with the spherical bearing surface and screwing bone screw 90 in the other fractured vertebral part to fix the bone plate on the other fractured vertebral part.

The mechanism for osteosynthesis of this embodiment can be preferably used for fastening fractured vertebra. By adjusting the length of the fractured bone by rotating the pinion pin 4, it is made easy to reduce the fractured bone. In addition, it is made easier to quantify the amount of movement of the fractured bone. Also because the pinion pin 4 is rotated with a wrench, the pinion pin 4 can be easily rotated even when the incision opening is small. Also the present invention is applicable to both pulling the bone fragments apart from each other and pulling the bone fragments nearer toward each other simply by changing the direction of rotating the pinion pin 4.

### Tenth Embodiment 10

FIG. 6I shows a mechanism for osteosynthesis 100I to be used in epiphysiys, where the epiphysial fixing portion 23 of the bone plate 2 is fixed onto the epiphysial part of the fractured bone and the diaphyseal fixing portion 21 of the bone plate 2 is fixed onto the diaphysis part of the fractured bone of the humerus. The mechanism for osteosynthesis 100I is significantly different from those of the first to ninth embodiments in that the sliding elongate hole 3 of the bone plate 2 is formed in the epiphysial fixing portion 23. The pinion pin 4 is disposed in the sliding elongate hole 3, and the holes 50 with the spherical bearing surface are formed on either side of the sliding elongate hole. A plurality of female-threaded holes 5 is formed in the diaphyseal fixing portion 21. The mechanism for osteosynthesis shown in FIG. 6I is used in case the epiphysial part has been dislocated laterally with respect to the diaphyseal part. The epiphysial part can be moved laterally with respect to the diaphyseal part by means of a sliding part.

Method of using the mechanism for osteosynthesis 100I comprises (1) fixing the diaphyseal fixing portion 21 of the bone plate 2 on the diaphyseal part of the fractured bone; (2) inserting the pinion pin 4 in the sliding elongate hole 3 and in the epiphysial part to fix the bone plate on the epiphysial part of the fractured bone; (3) rotating the pinion pin 4 so as to move the epiphysial part laterally with respect to the diaphyseal part to reduce the fractured bone; and (4) inserting the bone screw 90 with the tapered head in the hole 50 with the spherical bearing surface and screwing bone screw 90 in the epiphysial part to fix the bone plate on the epiphysial part.

The mechanism for osteosynthesis of this embodiment is suited for fixing fractured bone in case the epiphysial part has been dislocated laterally. By moving the fractured bone that has been laterally dislocated to a predetermined position by rotating the pinion pin 4, it is made possible to achieve reduction and fixation of the fractured bone at the same time. In addition, it is made easier to quantify the amount of movement of the fractured bone. Also because the pinion pin 4 is rotated with a wrench, the pinion pin 4 can be easily manipulated and rotated even when the incision opening is small.

### Modification Examples

In a modification example of the tenth embodiment, such a mechanism for osteosynthesis may be used as the sliding elongate hole 3 of the bone plate is formed in an arc shape. This mechanism for osteosynthesis is useful, not only in a case where the epiphysial part of the fractured bone has been dislocated laterally, but also in such a case as the fractured bones have been dislocated in the direction of reducing the bone length (shortening dislocation) or the fractured bones have dislocated in the direction of departing from each other to widen a gap (separating dislocation). In a case of fracture where the bone has undergone shortening dislocation and lateral dislocation at the same time, for example, the sliding elongate hole 3 may have an arc shape with the center lying in the diaphysis. The sliding elongate hole 3 of such a configuration causes the epiphysial part to move along the arc-shaped path of the sliding elongate hole 3, so that the epiphysial part can be moved laterally away from the diaphyseal part of the fractured bone to achieve reduction. In a case of fracture where the bone has undergone separating dislocation and lateral dislocation at the same time, on the other hand, the sliding elongate hole 3 may have an arc shape with the center lying in the epiphysial part. The sliding elongate hole 3 of such a configuration causes the epiphysial part to move along the arc-shaped path of the sliding elongate hole 3, so that the epiphysial part can be moved laterally toward the diaphyseal part to achieve reduction.

### Eleventh Embodiment

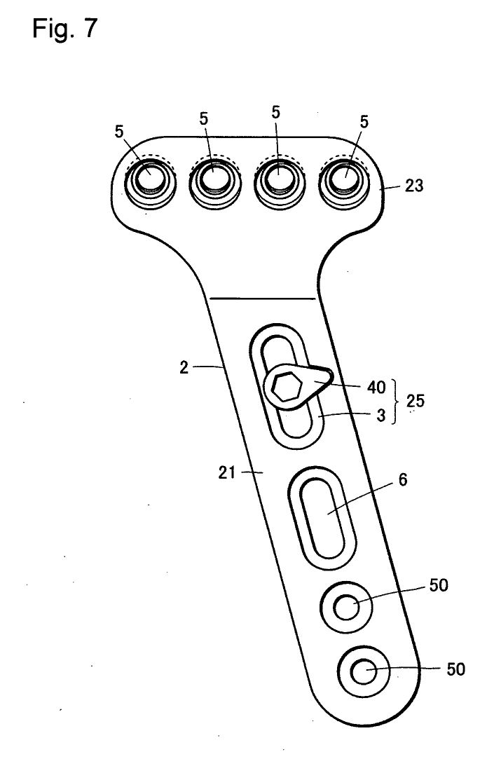

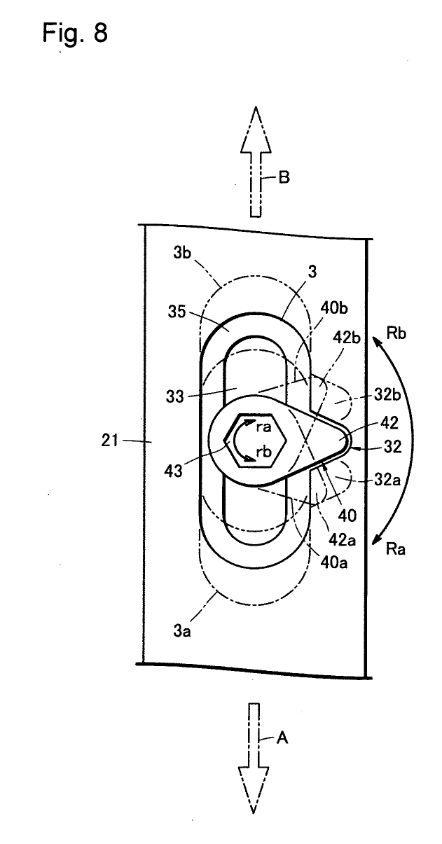

The mechanism for osteosynthesis 1 of this embodiment to be used in the epipysis of radius, shown in FIG. 7 and FIG. 8, is similar to that of the first embodiment except for using a cam mechanism in the sliding part 25.

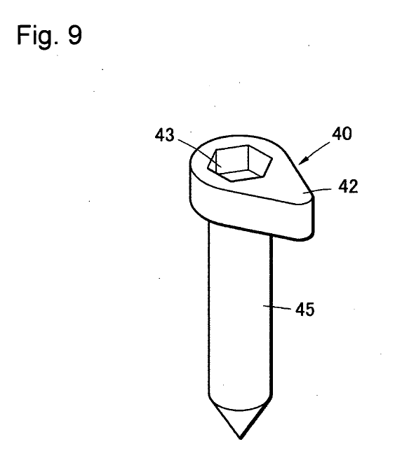

The sliding elongate hole 3 of the bone plate 2 has a cam receiving portion 32 having concave shape formed on one of longer sides thereof extending in the sliding direction. The mechanism for osteosynthesis 1 also includes a pin (cam pin 40) comprising a head (a cam portion 42) of eccentric configuration that is inserted into the sliding elongate hole 3 and is slidably fitted with the cam receiving portion 32 and a base portion (a shaft 45) to be inserted into the bone. The sliding part 25 is constituted by combining the sliding elongate hole 3 and the cam pin 40. The cam pin 40 is fixed onto the bone by inserting the shaft 45 in one fractured bone whereon the sliding elongate hole 3 has been aligned. As shown in FIG. 9, the cam pin 40 has the cam portion 42 of eccentric configuration in the head portion thereof, so that a protruding portion of the cam portion 42 engages slidably with the cam receiving portion 32 of the sliding elongate hole 3. Lower part of the cam pin 40 is formed as a cylindrical shaft 85 of which a tip is formed in a conical shape.

The sliding part 25 constituted from the sliding elongate hole 3 and the cam pin 40 will be described in detail with reference to FIG. 8.

The cam portion 42 of the cam pin 40 and the cam receiving portion 32 of the sliding elongate hole 3 engage with each other in the sliding elongate hole 3. The shaft 45 of the cam pin 40 is inserted in one (the diaphyseal part) of the fractured bones in advance. As the cam pin 40 is rotated in the direction indicated by arrow rₐ, the protruding portion of the cam portion 42 rotates in the direction indicated by arrow Rₐ while sliding in the recess of the cam receiving portion 32, so that the protruding portion of the cam portion 42 moves to a position indicated by an alternate dot and dash line in the drawing (a cam portion 42a) while rotating. Accordingly, the cam receiving portion 32 is pressed in the direction of arrow A (downward in the drawing) and the diaphyseal fixing portion 21 of the mechanism for osteosynthesis 1 also moves in the direction of arrow A. When the cam pin 40 of this mechanism for osteosynthesis 1 is rotated in the direction indicated by arrow r_{b}, the protruding portion of the cam portion 42 rotates in the direction indicated by arrow R_{b} while sliding in the recess of the cam receiving portion 32, so that the protruding portion of the cam portion 42 moves to a position indicated by an alternate two dots and dash line in the drawing (a cam portion 42b) while rotating. Accordingly, the cam receiving portion 32 is pressed in the direction of arrow B (upward in the drawing) and the diaphyseal fixing portion 21 of the bone plate 2 also moves in the direction of arrow B. The mechanism for osteosynthesis 1 that employs the cam mechanism in the sliding part 25 is capable of reducing the fractured bone in this way. The cam pin 40 has the hexagonal socket 43 formed at the center of the head thereof, and is rotated by means of a hexagonal wrench that matches the hexagonal socket 43.

The present invention has the constitution provided with the cam pin 40 and the sliding elongate hole 3, and thereby enables it to reduce fractured bone by rotating the cam pin 40. This makes it easier to reduce the fractured bone and makes it possible to easily quantify the amount of movement of the fractured bone. Also because the cam pin 40 can be rotated by means of a wrench, the operation of rotating the cam pin 40 is made easier and the reduction of the fractured bone can be done reliably even when the incision opening is small. The present invention can be applied to both pulling the bone fragments apart from each other and pulling the bone fragments nearer toward each other simply by changing the direction of rotating the cam pin 40.

1-8. (canceled)

9. A mechanism for osteosynthesis comprising:
a plate part for reducing two or more fractured bones monolithically by fixing at least both ends of the plate part on the fractured bones; and
a sliding part for moving one fractured bone along a sliding elongate hole formed in the plate part so as to elongate from the one fractured bone toward another fractured bone,
the sliding part comprising:
a rack formed on an inner surface of the sliding elongate hole so as to extend along a sliding direction; and
a pin inserted in the sliding elongate hole, the pin having a head portion having a pinion for engaging with the rack and a base portion to be inserted into the one fractured bone,
wherein the sliding part moves the one fractured bone slidably along the sliding elongate hole by rotating the pin.

10. A mechanism for osteosynthesis comprising:
a plate part for reducing two or more fractured bones monolithically by fixing at least both ends of the plate part on the fractured bones; and
a sliding part for moving one fractured bone along a sliding elongate hole formed in the plate part so as to elongate from the one fractured bone toward another fractured bone,
the sliding part comprising:
a cam receiving portion formed on an inner surface of the sliding elongate hole; and
a pin inserted in the sliding elongate hole, the pin having a head portion having a cam for engaging with the cam receiving portion and a base portion to be inserted into the one fractured bone,
wherein the sliding part moves the one fractured bone slidably along the sliding elongate hole by rotating the pin.

11. The mechanism for osteosynthesis according to claim 9, wherein an auxiliary sliding elongate hole for assisting a sliding motion is further formed in the plate part so as to elongate parallel to the sliding elongate hole.

12. The mechanism for osteosynthesis according to claim 10, wherein an auxiliary sliding elongate hole for assisting a sliding motion is further formed in the plate part so as to elongate parallel to the sliding elongate hole.

13. The mechanism for osteosynthesis according to claim 9, wherein the plate part has a female-threaded hole having a female thread,
the plate being fixed on the fractured bone by inserting the bone pin or the bone screw in the female-threaded hole, the bone pin and the bone screw having a head portion having a male thread for mating with the female thread and a base portion to be inserted into the fractured bone.

14. The mechanism for osteosynthesis according to claim 10, wherein the plate part has a female-threaded hole having a female thread,
the plate being fixed on the fractured bone by inserting the bone pin or the bone screw in the female-threaded hole, the bone pin and the bone screw having a head portion having a male thread for mating with the female thread and a base portion to be inserted into the fractured bone.

15. The mechanism for osteosynthesis according to claim 9, wherein the plate part has a hole with a spherical bearing surface,
the plate being fixed on the fractured bone by inserting the bone screw in the hole, the bone screw having a head portion capable of swinging on the spherical bearing surface and a base portion to be inserted into the fractured bone.

16. The mechanism for osteosynthesis according to claim 10, wherein the plate part has a hole with a spherical bearing surface,
the plate being fixed on the fractured bone by inserting the bone screw in the hole, the bone screw having a head portion capable of swinging on the spherical bearing surface and a base portion to be inserted into the fractured bone.

17. The mechanism for osteosynthesis according to claim 1, wherein the mechanism is used for any one of regions of humerus, radius, ulna, vertebra, femur, tibia, fibula, phalanges of hand and phalanges of foot.

18. The mechanism for osteosynthesis according to claim 10, wherein the mechanism is used for any one of regions of humerus, radius, ulna, vertebra, femur, tibia, fibula, phalanges of hand and phalanges of foot.

19. A bone plate for moving one of two or more fractured bones toward another fractured bone, the bone plate including a plate part for reducing two or more fractured bones monolithically by fixing at least both ends of the plate part on the fractured bones,
the bone plate comprising:
a sliding elongate hole elongating from the one fractured bone toward the another fractured bone; and
a rack formed on an inner surface of the sliding elongate hole so as to extend along a sliding direction,
wherein the bone plate enables to constitute a sliding part by inserting a pin in the sliding elongate hole, the pin having a head portion having a pinion for engaging with the rack and a base portion to be inserted into the one fractured bone, the sliding part moving the one fractured bone slidably by rotating the pin.

20. A pin to be inserted in a sliding elongate hole which is formed in a plate part for reducing two or more fractured bones monolithically and elongates from one fractured bone toward another fractured bone,
the pin constituting a sliding part together with a rack which is formed on an inner surface of the sliding elongates hole and extends along a longitudinal direction of the sliding elongate hole,
the pin comprising a head portion having a pinion for engaging with the rack and a base portion to be inserted into the one fractured bone,
wherein the sliding part moves the one fractured bone slidably along the sliding elongate hole by rotating the pin.
